(19) United States
(12) Patent Application Publication
(10) Pub. No.: US 2010/0056981 A1
(43) Pub. Date: Mar. 4, 2010

(54) Tampon Having An Improved Finger Pocket

(76) Inventors: Nancy Karapasha, Cincinnati, OH (US); Matthew Howard Wasson, Cincinnati, OH (US); Amaranta Ramirez-Almaraz, Cincinnati, OH (US); Holger Wendt, Budapest (HU)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/198,154

(22) Filed: Aug. 26, 2008

(51) Int. Cl. A61F 13/20; A61F 13/15

(52) U.S. Cl. 604/11; 604/385.18; 604/385.17; 28/120

(57) ABSTRACT

A tampon having an improved finger pocket. The finger pocket can have a width that can be calculated by the equation D_{F}=D_{T}−(L₁+L₂), where D_{F} is the width of the finger pocket, D_{T} is the diameter of the tampon, L₁ is the thickness of the steering lip on a first side and L₂ is the thickness of a steering lip on a second side, and/or a ratio of the indentation width to the base diameter from about 1:1.1 to about 1:1.5. In certain embodiments, the base can have at least one steering lip adjacent the indention that can have a thickness of at least about 0.5 mm. A method for making a tampon having an improved finger pocket that can include forming a compressed pledget having an insertion end and a base, and compressing the base of the pledget with a heated compression member to form an improved finger pocket in the base.

### FIELD OF THE INVENTION

This invention relates to tampons, and more particularly to tampons having an improved finger pocket.

### BACKGROUND OF THE INVENTION

Catamenial tampons are typically inserted into a vagina of a woman to absorb menstrual discharges during menstruation. Such tampons are generally inserted either with the aid of an applicator or digitally via a user's finger. In the case of a digital tampon, the user may unwrap the tampon, extend the withdrawal member, and grasp the tampon, such as, for example, by pushing her finger or nail into the base of the tampon. The user can then insert the tampon into her body and steer the tampon with her finger to the desired location.

Current digital tampons, however, do not have a well-defined place for a user to insert her finger into the base of the tampon. For example, some digital tampons do not have a preformed finger pocket in the base at all. Instead, these digital tampons may have a less compressed base that the user can push her finger into. This can result in a user pushing her finger or nail into the tampon at a different location each time she inserts a tampon, which can prevent the user from achieving a consistent usage and placement experience. Other digital tampons can have a finger pocket, but the finger pocket may not be well-defined or ergonomically configured such that the user's insertion experience and placement is consistent from tampon to tampon, even, for example, across tampon sizes.

As such, it would be desirable to provide a tampon having improved insertion control, such as, e.g., a tampon with an improved finger pocket. It would also be desirable to provide a process for forming such an improved finger pocket.

### SUMMARY OF THE INVENTION

A tampon having an improved finger pocket is provided. The tampon can have an insertion end, a base, and a length measured from the insertion end to the base parallel to a longitudinal axis of the tampon. The base can have a diameter, an edge, and a generally flattened surface having an indentation configured to accommodate at least a portion of a user's finger. The indentation can have a width measured in a plane of the generally flattened surface and a height measured perpendicularly to the width. The base can have at least one steering lip adjacent the indention. The lip can have a thickness measured in the plane of the generally flattened surface from the indention to the edge. The finger pocket width can be calculated by the equation DF=DT−(L1+L2), where DF is the width of the finger pocket, DT is the diameter of the tampon, L1 is the thickness of the steering lip on a first side and L2 is the thickness of a steering lip on a second side.

Also provided is a tampon having an improved finger pocket that has an insertion end, a base, and a length measured from the insertion end to the base parallel to a longitudinal axis of the tampon. The base can have a diameter, an edge, and a generally flattened surface having an indentation configured to accommodate at least a portion of a user's finger. The indentation can have a width measured in a plane of the generally flattened surface and a height measured perpendicularly to the width. In certain embodiments, a ratio of the indentation width to the base diameter can be from about 1:1.1 to about 1:1.5.

Also provided is a method for making a tampon having an improved finger pocket. The method can include forming a compressed pledget having an insertion end and a base, and compressing the base of the pledget with a heated compression member to form an improved finger pocket in the base.

### BRIEF DESCRIPTION OF THE DRAWINGS

FIG. 1 is a perspective view of a tampon.

FIG. 2 is an end view of a tampon.

FIG. 3 is a plan view of a tampon.

FIG. 4 is a plan view of a tampon forming apparatus.

### DETAILED DESCRIPTION OF THE INVENTION

The present invention relates to tampons having an improved finger pocket. It has now been discovered that such tampons can provide a more secure insertion experience to a user, such as, for example, more precise insertion control, reliable placement, and/or a reproducible and/or confident experience. In certain embodiments, the finger pocket can have a predefined width and height and can be bounded by at least one steering lip. In addition, in certain embodiments, the predefined width can vary with the diameter of the tampon base, such as, for example, as a ratio of predefined width of the finger pocket to diameter of the tampon base that can, in certain embodiments, remain substantially the same across varying tampon sizes and/or absorbencies. Furthermore, in certain embodiments, the finger pocket can have walls that define an area that has a volume and the volume can be substantially free and/or generally free of fiber.

A. Definitions

As used herein, the term “absorbency level” refers to the grams of fluid that typically can be absorbed by a tampon. Any suitable classification for absorbency level, such as, e.g., past, current, and future U.S., European, or other classification schemes, can be used. One example of a absorbency level classification scheme includes, e.g., a junior or light absorbency level corresponding to a tampon absorbency less than or equal to 6 g of fluid as measured by the syngyna test, a regular absorbency level generally corresponding to 6 g to 9 g of fluid, a super absorbency level generally corresponding to 9 g to 12 g of fluid, a super plus absorbency level generally corresponding to 12 g to 15 g of fluid, and an ultra absorbency level generally corresponding to 15 g to 18 g of fluid as measured by the syngyna test. Another example of an absorbency level classification scheme includes, e.g., one droplet corresponding to a tampon absorbency level less than or equal to 6 g of fluid as measured by the syngyna test, two droplets corresponding to 6 g to 9 g of fluid, three droplets corresponding to 9 g to 12 g of fluid, four droplets corresponding to 12 g to 15 g of fluid, five droplets corresponding to 15 g to 18 g of fluid, and six droplets corresponding to 18 g to 21 g of fluid as measured by the syngyna test.

As used herein, “actuating” is any force delivered by an electric motor, mechanical transmission, pneumatical, linear drive, manual, and/or hydraulic.

As used herein, “compression” refers to the process of pressing, squeezing, compacting or otherwise manipulating the size, shape, and/or volume of a material to obtain a tampon having a vaginally insertable shape. The term “compressed” refers to the state of a material or materials subsequent to compression. Conversely, the term “uncompressed” refers to the state of a material or materials prior to compression. The term “compressible” is the ability of a material to undergo compression.

As used herein, the “compression member” is any member that can be used to compress a pledget. It can also function to transfer a compressed pledget. In certain embodiments, the compression member can function as a pocket forming member.

As used herein, the term “density” is used with its common technical meaning with units of g/cm³ or g/cc. The density may refer specifically to that of a specific region or feature of the tampon as noted. The density will be measured, unless otherwise noted, by taking the weight divided by the geometric volume described by the shape. Unless noted, density refers to that of the overall structure and not the individual components, and will include in the measurement void volume of small pores and voids within the overall structure.

As used herein, the term “insertion end” refers to the portion of the tampon or applicator including the end that is intended to enter the vaginal canal first when inserting the tampon or applicator into the vaginal canal.

As used herein, “mold” refers to a structure for supporting and/or shaping a pledget during compression, header and/or finger pocket formation, and/or during the stabilization process.

As used herein the term “patterned impression” refers to a shape imparted to a surface by compressing material with a die. The shape imparted comprises one or more design elements corresponding to die elements of the die. Patterns may include a wide variety of design elements including but not limited to dimples, dots, concentric circles, cross-hatching, stars, polygons, arcs, diamonds, triangles, rings, lines, rectangles, petals, ellipsoids, tear drops, letters, or waves. In certain embodiments, the pattern can include design elements, such as, e.g., recesses and/or protrusions, that can be separated by land areas between the recesses and/or protrusions.

As used herein the term “pledget” refers to a construction of absorbent material prior to the compression of such construction into a tampon.

As used herein, the “pocket forming member” is any member that can be used to form the finger pocket. In certain embodiments, the pocket forming member can be the compression member. In addition, or alternatively, the pocket forming member can be heated. In certain embodiments, the pocket forming member can be patterned, such as, e.g., to provide one or more patterned impressions within the finger pocket.

As used herein, “self-sustaining” is a measure of the degree or sufficiency to which the tampon retains its compressed form after stabilization such that in the subsequent absence of external forces, the resulting tampon will tend to retain its vaginally insertable shape and size. It will be understood by one of skill in the art that this self-sustaining form need not, and may not persist during actual use of the tampon. That is, once the tampon is inserted into the vagina or other body cavity and begins to acquire fluid, the tampon will begin to expand and may lose its self-sustaining form.

The term “stabilized,” as used herein, refers to a tampon in a self-sustaining state wherein it has overcome the natural tendency to re-expand to the original size, shape and volume of the absorbent material and overwrap, which comprise the pledget.

As used herein, the term “tampon,” refers to any type of absorbent structure that is inserted into the vaginal canal or other body cavity for the absorption of fluid therefrom, to aid in wound healing, or for the delivery of active materials, such as medicaments, or moisture. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as oval, rectangular, triangular, trapezoidal, semi-circular, hourglass, serpentine, or other suitable shapes. Tampons have an insertion end, withdrawal end, a length, a width, a longitudinal axis and a radial axis. The tampon's length can be measured from the insertion end to the withdrawal end along the longitudinal axis. A typical compressed tampon for human use is within a range from about 30 mm to about 60 mm in length. A tampon may be straight or non-linear in shape, such as curved along the longitudinal axis. A typical compressed tampon is within a range from about 8 mm to about 20 mm wide. The width of a tampon, unless otherwise stated in the specification, corresponds to the distance across the largest cross-section, along the length of the tampon and perpendicular to the longitudinal axis of the tampon.

As used herein, the “tampon compression machine” is a machine assembly that includes parts that may compress a pledget. Typically a pledget compressed in the tampon compression machine is then transferred to a mold for final shaping into a self-sustaining form of a vaginally insertable shape where the mold may further compress parts of the pledget beyond that which the tampon compression machine accomplished prior.

The term “vaginal cavity,” “within the vagina,” and “vaginal interior,” as used herein, are intended to be synonymous and refer to the internal genitalia of the mammalian female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

As used herein, the term “withdrawal end” refers to the portion of the tampon or applicator opposite the insertion end.

B. Tampons

Tampons made according to certain embodiments of this invention may have an improved finger pocket. In certain embodiments, the finger pocket can have a predefined width and height and can be bounded by at least one steering lip. In addition, the finger pocket can have walls that define an area that has a volume and the volume can be substantially free and/or free of fiber.

FIG. 1 shows a compressed tampon 10 having an insertion end 11, a base 12 opposite the insertion end 11, and a length L measured between the insertion end 11 and the base 12 parallel to a longitudinal axis (l) of the tampon 10 and a width extending substantially perpendicularly to the length of the tampon from a first side 13 to a second side 14. In addition, the base 12 has a diameter D and includes a finger pocket 15 disposed to accommodate at least a portion of a user's finger. The finger pocket 15 has a width W measured along the plane of the base 12. As shown in FIG. 1, the base 12 can have a steering lip 16.

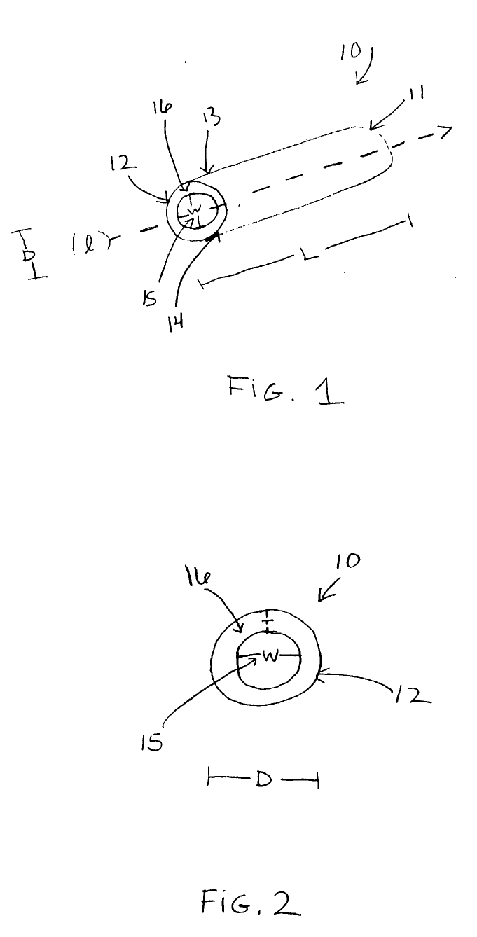

FIG. 2 shows an end view of the tampon 10 of FIG. 1. The tampon 10 has a finger pocket 15 provided in the base 12. The finger pocket 15 can be configured to accommodate at least a portion of a user's finger during insertion of the tampon 10. As shown in FIG. 2, the base 12 has a diameter D and the finger pocket 15 has a width W. The finger pocket 15 can be bounded by one or more steering lips 16. In certain embodiments, the finger pocket 15 can be bounded by one or more steering lips 16 that can substantially surround the finger pocket 15. As shown in FIG. 2, the steering lips 16 can have a thickness T.

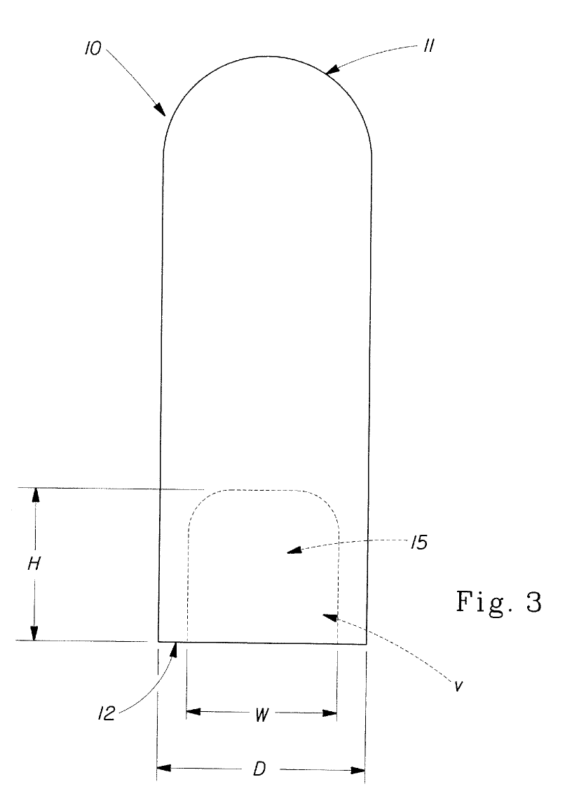

FIG. 3 shows a tampon 10 having a finger pocket 15. The tampon 10 has an insertion end 11, a base 12 disposed opposite the insertion end 11, and a finger pocket 15 provided in the base 12. The base 12 has a diameter D. The finger pocket 15 can have a height H, a width W, and a volume V.

The inventive tampons have an improved finger pocket that can be a predetermined and well-defined configuration. In certain embodiments, the finger pocket is a predetermined size and/or location and is configured to accommodate at least a portion of a user's finger, such as, for example, a portion of a user's finger sufficient to retain the tampon on the user's finger after insertion of the finger into the finger pocket. In certain embodiments, the finger pocket can be configured to cover a portion of a user's finger at least about 25%, about 50%, or more down the length of the user's nail bed when the tampon is placed on the user's fingertip.

The finger pocket will typically be a size and configuration suitable for providing the improved benefits described herein. For example, it has been discovered that suitable finger pocket configurations calculated by the following equation can provide improved stability and insertion control to a user:

D_{F}=D_{T}−(L₁+L₂)

where D_{F} is the width of the finger pocket, D_{T} is the diameter of the tampon, L₁ is the thickness of the steering lip on a first side and L₂ is the thickness of a steering lip on a second side. In certain embodiments, the steering lip can be the same thickness on either side of the finger pocket, in which case, the equation would be:

D_{F}=D_{T}−(L×2)

where D_{F} is the width of the finger pocket, D_{T} is the diameter of the tampon, and L₁ is the thickness of the steering lip. In certain embodiments, D_{F} can vary by from about ±1% to about ±15%, such as, e.g., from about ±5% to about ±10%. This variation is thought to be related to the rebound demonstrated by tampon fibers after compression. In certain embodiments, L can be greater than zero, L₁ can be greater than zero, and/or L₂ can be greater than zero.

In certain embodiments as described herein, the finger pocket can have a ratio of finger pocket width to tampon base diameter of from about 1:1.1 to about 1:1.5, such as, e.g., about 1:1.2 to about 1:1.4, such as, e.g., about 1:1.2 or 1:1.3. In addition, the finger pocket can be bounded by at least one steering lip, such as, e.g., a steering lip having a thickness of from about 0.5 mm to about 2 mm, such as, e.g., from about 1 mm to about 1.5 mm. Example tampons having a suitable finger pocket size and ratio of finger pocket width to tampon base diameter are described in Table 1:

TABLE 1
Ratio of Finger Pocket Width to Tampon
Base Diameter With Varying Absorbencies

| Absorbency Size | | Base Diameter | Finger Pocket Width | Ratio |
|---|---|---|---|---|
| <6 | g | 9.4 mm | 7.4 mm | 1.27 |
| 6-9 | g | 10.4 mm | 8 mm | 1.30 |
| 9-12 | g | 11.4 mm | 9 mm | 1.27 |
| 12-15 | g | 12.4 mm | 10 mm | 1.24 |
| 15-18 | g | 13.4 mm | 11 mm | 1.22 |

As shown in Table 1, the ratio of finger pocket width to tampon base diameter can stay substantially the same or completely the same across different sizes and/or absorbencies. Finger pocket width and/or tampon base diameter can be measured at any suitable time after finger pocket formation, such as, e.g., when the tampon is removed from the mold, at about one month after packaging, at about three months after packaging, at about six months after packaging, or at about one year after packaging or more.

In certain embodiments, the finger pocket can be bounded by at least one steering rim. Such steering rims can have a thickness that can be suitable to assist a user in grasping the tampon for insertion into her body. The at least one steering rim can bound the finger pocket on any or all sides, such as, e.g., on one side, on two sides, on three sides, on four sides, and/or can substantially or completely surround the finger pocket, such as, e.g., by being adjacent the finger pocket. In addition, in certain embodiments, the steering rim can be provided adjacent and between the finger pocket and edge of the tampon base. The steering rim can be any suitable thickness, such as, for example, at least about 0.5 mm, such as, e.g., from about 0.5 mm to about 2 mm, from about 1 mm to about 1.5 mm, or any other suitable thickness.

The finger pocket can be any suitable shape and/or cross-section, such as, e.g., a shape suitable for accommodating at least a portion of a user's finger. Suitable shapes can include, for example, circular, oval, elliptical, rectangular, square with rounded corners, diamond, domed, cylindrical, conical, tapered, pointed, or any other suitable shape. In certain embodiments, the finger pocket can generally have a circular shape and/or cross-section.

The finger pocket can have a predetermined volume, such as, e.g., a volume suitable for accommodating at least a portion of a user's finger. In certain embodiments, the predetermined volume of the improved finger pocket can be substantially or generally free of fiber, such as, e.g., substantially or generally free of fibers from the tampon pledget.

In addition, in certain embodiments, the finger pocket can be patterned, such as, e.g., a finger pocket having one or more patterned impressions. The patterned impressions can, for example, provide finger stabilization and/or improved gripping and/or steering. Suitable patterns can include, for example, dimples, dots, concentric circles, cross-hatching, stars, polygons, arcs, diamonds, triangles, rings, lines, rectangles, petals, ellipsoids, tear drops, letters, or waves. In certain embodiments, the patterned impressions can be convex. The patterned impressions can be disposed over substantially the entire surface of the finger pocket, or can be disposed over a portion of the surface of the finger pocket. In certain embodiments, the surface of the finger pocket can have more than one type of patterned impression that can be provided in predetermined locations on the surface of the finger pocket, such as, e.g., a first patterned impression that can be provided to improve steering and a second patterned impression that can be provided to orient the user's finger, or any other suitable combination of patterned elements.

In certain embodiments, the tampon can have a length extending between the withdrawal end and the insertion end. In such an embodiment, the tampon may have a patterned impression in the withdrawal end, the insertion end, or both. In addition, the body portion of the tampon may have substantially no patterned impression. According to certain embodiments, a tampon with a patterned end may have a body portion with creases formed during compression of the uncompressed pledget, but such creases may not be considered a patterned impression. In addition, the patterned impression can be one or more colors and/or can include one or more design elements and the one or more design elements can be the same or different colors.

The absorbent material of the tampon may be constructed from a wide variety of liquid absorbing materials commonly used in absorbent articles. Such materials include but are not limited to rayon (such as GALAXY rayon, SARILLE L rayon both available from Kelheim Fibres GmbH of Kelheim, Germany), cotton, folded tissues, woven materials, nonwoven webs, synthetic and/or natural fibers or sheathing, comminuted wood pulp which is generally referred to as airfelt, or combinations of these materials. Other materials that may be incorporated into the pledget 12 include peat moss, absorbent foams (such as those disclosed in U.S. Pat. No. 3,994,298 issued to Desmarais on Nov. 30, 1976 and U.S. Pat. No. 5,795,921 issued to Dyer, et al.), capillary channel fibers (such as those disclosed in U.S. Pat. No. 5,356,405 issued to Thompson, et al. issued on Oct. 18, 1994), high capacity fibers (such as those disclosed U.S. Pat. No. 4,044,766 issued to Kaczmarck, et al. on Aug. 30, 1994), and super absorbent polymers or absorbent gelling materials (such as those disclosed in U.S. Pat. No. 5,830,543 issued to Miyake, et al. on Nov. 3, 1998). A more detailed description of liquid absorbing materials can be found in U.S. Pat. No. 6,740,070 to Raymond Agyapong.

The tampon may optionally include an overwrap comprising materials such as rayon, cotton, bicomponent fibers, polyethylene, polypropylene, other suitable natural or synthetic fibers known in the art, and mixtures thereof. In some embodiments, the tampon may have a nonwoven overwrap comprised of bicomponent fibers that have a polypropylene core surrounded by polyethylene manufactured by Vliesstoffwerke Christian Heinrich Sandler GmbH and Company KG (Schwarzenbach/Salle Germany) under the trade name SAS B31812000. In certain embodiments, the tampon may comprise a nonwoven overwrap of a hydro entangled blend of 50% rayon, 50% polyester available as BBA 140027 produced by BBA Corporation of South Carolina, US. In certain embodiments, the overwrap may be treated to hydrophilic, hydrophobic, wicking or nonwicking. In certain embodiments, the tampon may comprise an overwrap of formed film. The tampon, in certain embodiments, may include an overwrap covering the entire tampon or only a portion of the tampon.

The tampon can include a withdrawal cord attached to the tampon that can be made of any suitable material, such as, for example, cotton and rayon. In certain embodiments, the tampon may include a secondary absorbent member, an additional overwrap, a skirt portion and/or an applicator. U.S. Pat. No. 6,258,075 issued to Taylor et al. describes a variety of secondary absorbent members for use in pledgets. An example of a skirt portion is disclosed in U.S. Pat. No. 6,840,927 to Margaret Hasse.

C. Tampon Manufacturing Apparatus

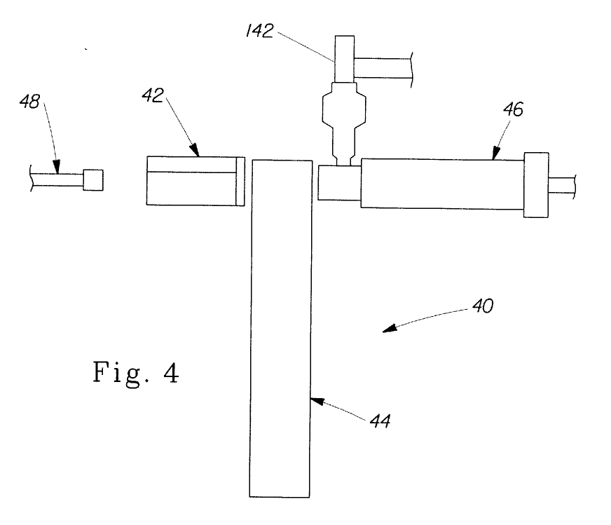

Turning to FIG. 4, a tampon forming apparatus 40, in accordance with certain embodiments of this invention, is illustrated. In certain embodiments, the tampon forming apparatus 40 may generally comprise a compression machine 42, a mold 44, and a header assembly 46. The mold 44 may be disposed between the compression machine 42 and the header assembly 46. The tampon forming apparatus 40 may also include a compression member 48. In certain embodiments, however, the tampon forming apparatus 40 need not include a compression machine 42 or a header assembly 46.

In certain embodiments, the compression machine 42, if present, may be configured for receiving an uncompressed pledget. The compression machine 42 can then compress the uncompressed pledget into a compressed pledget in any suitable manner, such as, e.g., widthwise, lengthwise, radially, or any other suitable configuration, after which the compressed pledget can be transferred to mold 44. Alternatively, the uncompressed pledget can be compressed in mold 44 or in any other suitable manner. Once the pledget is disposed within mold 44, the pledget can be stabilized in any suitable manner, such as, e.g., by application of steam, microwave energy, or in any other suitable manner.

As shown in FIG. 4, the tampon forming apparatus 40 can, in certain embodiments, include a header assembly 46. In addition, the header assembly 46 can include a patterned die that can, for example, be configured to provide one or more patterned impressions on the insertion end of the tampon. The patterned die 136 may have any suitable configuration. According to certain embodiments, the patterned die 136 may be concave or dome-shaped and may comprise a plurality of die elements 140 which may be protrusions or recesses separated by land areas 141.

The tampon forming apparatus 40 includes a compression member 48. The compression member 48 can be configured to provide a finger pocket in the withdrawal end of the tampon. In certain embodiments, the compression member 48 can be heated. The head of the compression member 48 can be any shape suitable for forming the finger pocket, such as, for example, domed, flat, conical, tapered, pointed, or any other suitable shape. In addition, or alternatively, the compression member 48 can be patterned, such as, e.g., to provide one or more patterns within the finger pocket, such as, for example, on the surface of the finger pocket.

The manner of actuation of the parts of the tampon forming apparatus 40, such as, e.g., the header assembly 46 and the compression member 48 may be by any suitable means and in any suitable configuration. For example, in certain embodiments, the header assembly 46 and the compression member 48 can be configured to form a header and finger pocket at the same time with one or multiple actuations. Alternatively, the header assembly 46 and the compression member 48 can be configured to form a header and finger pocket at different times. In addition, in certain embodiments, the tampon forming apparatus 40 will not include a header assembly 46 and the compression member 48 can form a finger pocket with one or multiple actuations. Furthermore, in certain embodiments, the tampon forming apparatus 40 can include a separate pocket forming member that is distinct from the compression member 48.

D. Method of Making Tampons

According to certain embodiments, the tampon 10 may be made by first inserting an uncompressed pledget in compression machine 42. The uncompressed pledget may then be compressed in the compression machine 42 to form a compressed pledget. The compressed pledget can be transferred to mold 44 and the tampon can be stabilized. A header and/or finger pocket can be formed by actuation of compression member 48 and/or header assembly 46, either prior to, during, or after stabilization of the tampon. In certain embodiments, compression member 48 and/or header assembly 46 can be patterned. In addition, or alternatively, compression member 48 and/or header assembly 46 can be heated.

In certain embodiments, the method can include forming a compressed pledget having an insertion end and a base having a diameter, an edge, and a generally flattened surface disposed opposite the insertion end. The base of the pledget can be compressed with a heated compression member 48 to form an improved finger pocket in the generally flattened surface. The improved finger pocket can have a predetermined width measured in a plane of the generally flattened surface and height measured perpendicularly to the width, and can be configured to accommodate at least a portion of a user's finger. In addition, the tampon can have a withdrawal member that can extend from the base of the tampon. In certain embodiments, the withdrawal member can be arranged about the base of the tampon prior to application of the compression member 48 and the compression member 48 can be applied to the base of the pledget over all or a portion of the withdrawal member to form the finger pocket. Alternatively, the withdrawal member can be arranged such that a majority of the withdrawal member is manipulated substantially away from the base of the tampon such that the compression member 48 can be applied directly to the base of the pledget to form the finger pocket.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior all with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon comprising an improved finger pocket, the tampon having an insertion end, a base, and a length measured from the insertion end to the base parallel to a longitudinal axis of the tampon, the base having a diameter, an edge, and a generally flattened surface having an indentation configured to accommodate at least a portion of a user's finger, the indentation having a width measured in a plane of the generally flattened surface and a height measured perpendicularly to the width, the base having at least one steering lip adjacent the indention, the lip having a thickness measured in a the plane of the generally flattened surface from the indention to the edge, the width being calculated by the equation D_{F}=D_{T}−(L₁+L₂), where D_{F} is the width of the finger pocket, D_{T} is the diameter of the tampon, L₁ is the thickness of the steering lip on a first side and L₂ is the thickness of a steering lip on a second side.

2. The tampon of claim 1, wherein the steering lip substantially surrounds the indentation.

3. The tampon of claim 1, wherein the indentation has a surface and the surface includes one or more patterned impressions.

4. The tampon of claim 1, wherein the insertion end includes one or more patterned impressions.

5. The tampon of claim 1, wherein the width is from about 5 mm to about 15 mm.

6. The tampon of claim 1, wherein the thickness is at least about 0.5 mm.

7. The tampon of claim 1, wherein L₁ is substantially the same as L₂.

8. The tampon of claim 1, wherein D_{F} varies from about ±1% to about ±15%.

9. The tampon of claim 1, wherein the tampon has a syngyna absorbency from about 6 to about 9 grams and the indentation has a width of about 7 mm, the tampon has a syngyna absorbency from about 9 to about 12 grams and the indentation has a width of about 8 mm, the tampon has a syngyna absorbency from about 12 to about 15 grams and the indentation has a width of about 9 mm, the tampon has a syngyna absorbency from about 15 to about 18 grams and the indentation has a width of about 10 mm, or the tampon has a syngyna absorbency from about 18 to about 21 grams and the indentation has a width of about 11 mm.

10. The tampon of claim 1, wherein the indentation has a volume and the volume is substantially free of fiber.

11. A tampon comprising an improved finger pocket, the tampon having an insertion end, a base, and a length measured from the insertion end to the base parallel to a longitudinal axis of the tampon, the base having a diameter, an edge, and a generally flattened surface having an indentation configured to accommodate at least a portion of a user's finger, the indentation having a width measured in a plane of the generally flattened surface and a height measured perpendicularly to the width, wherein a ratio of the indentation width to the base diameter is from about 1:1.1 to about 1:1.5.

12. The tampon of claim 11, wherein the tampon is present in an array, the tampon having a first absorbency and being provided in an array with a second tampon that is a second absorbency, the second tampon having an insertion end, a base, and a length measured from the insertion end to the base parallel to a longitudinal axis of the second tampon, the base having a diameter, an edge, and a generally flattened surface having an indentation configured to accommodate at least a portion of a user's finger, the indentation having a width measured in a plane of the generally flattened surface and a height measured perpendicularly to the width, the second tampon having a ratio of the indentation width to the base diameter is from about 1:1.1 to about 1:1.5

13. The tampon of claim 11, wherein the base has at least one steering lip adjacent the indention, the lip having a thickness measured in a plane of the generally flattened surface from the indention to the edge, and wherein the thickness is at least about 0.5 mm.

14. The tampon of claim 11, wherein the indentation has at least one surface that includes one or more patterned impressions.

15. The tampon of claim 11, wherein the insertion end includes one or more patterned impressions.

16. The tampon of claim 11, wherein the indentation has a volume and the volume is substantially free of fiber.

17. A method for making a tampon having an improved finger pocket comprising:
forming a compressed pledget having an insertion end and a base having a diameter, an edge, and a generally flattened surface disposed opposite the insertion end; and
compressing the base of the pledget with a heated compression member to form an improved finger pocket in the generally flattened surface, the improved finger pocket having a predetermined width measured in a plane of the generally flattened surface and height measured perpendicularly to the width, the improved finger pocket being configured to accommodate at least a portion of a user's finger.

18. The method of claim 17, wherein a ratio of the finger pocket width to the base diameter is from about 1:1.1 to about 1:1.2.

19. The method of claim 17, wherein the base has at least one steering lip adjacent the finger pocket, the lip having a thickness measured in a plane of the generally flattened surface from the indention to the edge.

20. The method of claim 17, wherein the pledget includes a withdrawal member extending from the base, the withdrawal member being arranged about the base such that the heated compression member is applied to the base over at least a portion of the withdrawal member.
